(19) United States
(12) Patent Application Publication
(10) Pub. No.: US 2026/0345947 A1
(43) Pub. Date: Nov. 5, 2026

(54) THERAPEUTIC BREATHING APPARATUSES AND METHODS

(71) Applicants: Jennifer Hilderbrand, Queen Creek, AZ (US); Valerie Yepiz Lomeli, Queen Creek, AZ (US)

(72) Inventors: Jennifer Hilderbrand, Queen Creek, AZ (US); Valerie Yepiz Lomeli, Queen Creek, AZ (US)

(21) Appl. No.: 19/196,552

(22) Filed: May 1, 2025

(51) Int. Cl. A61M 21/02; A61M 21/00

(52) U.S. Cl. CPC A61M 21/02; A61M 2021/0088; A61M 2202/0007; A61M 2205/05; A61M 2205/583

(57) ABSTRACT

Disclosed herein are therapeutic breathing apparatuses and methods. One apparatus includes a circular structure including top and bottom surfaces including a perimeter and a diameter and a curved surface including a height that is less than the diameter extending between the perimeters. The apparatus further includes a breathing port for performing inhalations and exhalations a breathing pattern. One method includes using the apparatus to provide an indication of when to begin inhaling and provide another indication of when to cease inhaling and begin holding the user's breath. Another method includes sensing the presence of a user and illuminating visual cues on a breathing apparatus to notify the user to begin inhaling, illuminating the visual cues to notify the user to cease inhaling and begin holding the user's breath, and illuminating the plurality of visual cues to notify the user to cease holding the user's breath and begin exhaling.

### FIELD

The subject matter disclosed herein relates to breathing therapy and, more particularly, relates to therapeutic breathing apparatuses and methods.

### DESCRIPTION OF THE RELATED ART

Anxiety is a natural human response that is often felt when an individual believes they are under threat and is typically experienced through thoughts, feelings, and/or physical sensations. An individual can experience an excessive amount of anxiety in response to being overly worried, tense, and/or afraid; particularly when the individual strongly believes that something is about to happen or could happen in the future. As such, at least some individuals would benefit from new apparatuses and methods that can prevent an individual from experiencing an excessive amount of anxiety or at least reduce the amount anxiety the individual experiences in certain situations.

### BRIEF SUMMARY

Apparatuses and methods for therapeutic breathing are disclosed herein. One apparatus includes a circular structure including a top surface including a top perimeter and a diameter, a bottom surface including a bottom perimeter and the diameter, and a curved surface including a height extending between the top perimeter and the bottom perimeter in which the height of the curved surface is less than the diameter. The apparatus further includes a breathing port formed in the circular structure for performing inhalations and exhalations as portions of a breathing pattern.

One method includes providing a first indication, by a breathing apparatus, to a user to begin inhaling for a first predetermined amount of time as a first portion of a breathing pattern and providing a second indication, by the breathing apparatus, to the user to cease inhaling and begin holding the user's breath for a second predetermined amount of time as a second portion of the breathing pattern. The breathing apparatus includes a circular structure including a top surface including a top perimeter and a diameter, a bottom surface including a bottom perimeter and the diameter, and a curved surface including a height extending between the top perimeter and the bottom perimeter in which the height of the curved surface is less than the diameter. The breathing apparatus further includes a breathing port formed in the circular structure for performing inhalations and exhalations as portions of a breathing pattern in which the user inhales via the breathing port.

Another method includes sensing, by a sensor of a breathing apparatus, a presence of a user and in response to sensing the user, illuminating a plurality of visual cues on the breathing apparatus to notify the user to begin inhaling for a first predetermined amount of time as a first portion of a breathing pattern. The method further includes, in response to expiration of the first predetermined amount of time, illuminating the plurality of visual cues on the breathing apparatus to notify the user to cease inhaling and begin holding the user's breath for a second predetermined amount of time as a second portion of the breathing pattern and in response to expiration of the second predetermined amount of time, illuminating the plurality of visual cues on the breathing apparatus to notify the user to cease holding the user's breath and begin exhaling for a third predetermined amount of time as a third portion of the breathing pattern.

### BRIEF DESCRIPTION OF THE DRAWINGS

A more particular description of the embodiments briefly described above will be rendered by reference to specific embodiments that are illustrated in the appended drawings. Understanding that these drawings depict only some embodiments and are not therefore to be considered to be limiting of scope, the embodiments will be described and explained with additional specificity and detail through the use of the accompanying drawings, in which:

FIG. 1 is a schematic diagram of a top view of one embodiment of a therapeutic breathing device;

FIG. 2 is a schematic diagram of a bottom view of the therapeutic breathing device of FIG. 1;

FIG. 3 is a schematic diagram of a front view of the therapeutic breathing device of FIG. 1;

FIG. 4 is a schematic diagram of a rear view of the therapeutic breathing device of FIG. 1;

FIG. 5 is a schematic diagram of a first side view of the therapeutic breathing device of FIG. 1;

FIG. 6 is a schematic diagram of a second side view of the therapeutic breathing device of FIG. 1;

FIG. 7 is a schematic block diagram of one embodiment of circuitry included in the breathing device of FIG. 1; and

FIGS. 8 through 11 are schematic flow chart diagrams illustrating various embodiments of therapeutic breathing methods.

### DETAILED DESCRIPTION

As will be appreciated by one skilled in the art, aspects of the embodiments may be embodied as an apparatus or method. Accordingly, embodiments may take the form of an entirely hardware embodiment, an entirely software embodiment (including firmware, resident software, micro-code, etc.) or an embodiment combining software and hardware aspects that may all generally be referred to herein as a circuit, module, or apparatus. Furthermore, embodiments may take the form of a program product embodied in one or more computer-readable storage devices storing machine readable code, computer-readable code, and/or program code, referred hereafter as code. The storage devices may be tangible, non-transitory, and/or non-transmission. The storage devices may not embody signals. In a certain embodiment, the storage devices only employ signals for accessing code.

Code may include a single instruction, or many instructions, and may even be distributed over several different code segments, among different programs, and across several memory devices. Similarly, operational data may be identified and illustrated herein within modules and may be embodied in any suitable form and organized within any suitable type of data structure. The operational data may be collected as a single data set or may be distributed over different locations including over different computer-readable storage devices. Where a module or portions of a module are implemented in software, the software portions are stored on one or more computer-readable storage devices.

Any combination of one or more computer-readable media may be utilized. The computer-readable medium/media may include one or more computer-readable storage media. The computer-readable storage medium/media may be a storage device storing the code. The storage device may be, for example, but not limited to, an electronic, magnetic, optical, electromagnetic, infrared, holographic, micromechanical, or semiconductor system, apparatus, or device, or any suitable combination of the foregoing.

Code for carrying out operations for embodiments may be written in any combination of one or more programming languages including an object-oriented programming language such as Python, Ruby, Java, Smalltalk, C++, or the like, and conventional procedural programming languages, such as the C programming language, or the like, and/or machine languages such as assembly languages. The code may execute as a stand-alone software package.

Reference throughout this specification to one embodiment, an embodiment, or similar language means that a particular feature, structure, or characteristic described in connection with the embodiment is included in at least one embodiment. Thus, appearances of the phrases in one embodiment, in an embodiment, and similar language throughout this specification may, but do not necessarily, all refer to the same embodiment, but mean one or more but not all embodiments unless expressly specified otherwise. The terms including, comprising, having, and variations thereof mean including but not limited to, unless expressly specified otherwise. An enumerated listing of items does not imply that any or all of the items are mutually exclusive, unless expressly specified otherwise. The terms, “a,” “an,” and “the,” also refer to one or more unless expressly specified otherwise.

In addition, as used herein, the term, “set,” can mean one or more, unless expressly specified otherwise. The term, “sets,” can mean multiples of or a plurality of one or mores, ones or more, and/or ones or mores consistent with set theory, unless expressly specified otherwise.

Furthermore, the described features, structures, or characteristics of the embodiments may be combined in any suitable manner. In the following description, numerous specific details are provided, such as examples of programming, software modules, user selections, network transactions, database queries, database structures, hardware modules, hardware circuits, hardware chips, etc., to provide a thorough understanding of embodiments. One skilled in the relevant art will recognize, however, that embodiments may be practiced without one or more of the specific details, or with other methods, components, materials, and so forth. In other instances, well-known structures, materials, or operations are not shown or described in detail to avoid obscuring aspects of an embodiment.

Aspects of the embodiments are described below with reference to schematic flowchart diagrams and/or schematic block diagrams of apparatuses and methods according to embodiments. It will be understood that each block of the schematic flowchart diagrams and/or schematic block diagrams, and combinations of blocks in the schematic flowchart diagrams and/or schematic block diagrams, can be implemented by code. The code may be provided to a microcontroller, controller, processor of a general-purpose computer, special-purpose computer, or other programmable data processing apparatus to produce a machine, such that the instructions create means for implementing the functions/acts specified in the schematic flowchart diagrams and/or schematic block diagrams block or blocks.

The code may also be stored in a storage device that can direct a computer, other programmable data processing apparatus, or other devices to function in a particular manner, such that the instructions stored in the storage device produce an article of manufacture including instructions which implement the function/act specified in the schematic flowchart diagrams and/or schematic block diagrams block or blocks.

The code may also be loaded onto a computer, other programmable data processing apparatus, or other devices to cause a series of operational steps to be performed on the computer, other programmable apparatus or other devices to produce a computer implemented process such that the code which execute on the computer or other programmable apparatus provide processes for implementing the functions/acts specified in the flowchart and/or block diagram block or blocks.

The schematic flowchart diagrams and/or schematic block diagrams in the Figures illustrate the architecture, functionality, and operation of possible implementations of apparatuses, systems, methods and program products according to various embodiments. In this regard, each block in the schematic flowchart diagrams and/or schematic block diagrams may represent a module, segment, or portion of code, which includes one or more executable instructions of the code for implementing the specified logical function(s).

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and code.

The description of elements in each figure may refer to elements of proceeding figures. Like numbers refer to like elements in all figures, including alternate embodiments of like elements.

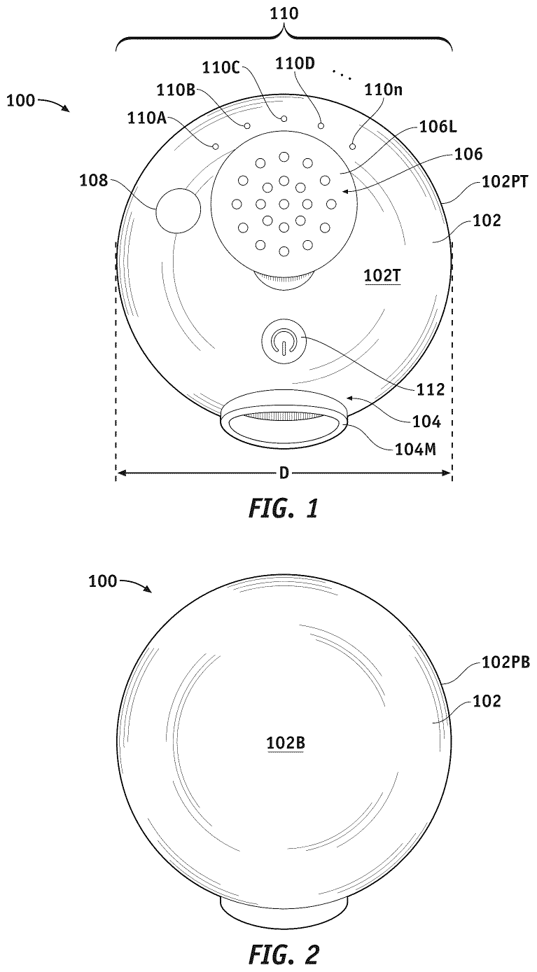

With reference to FIG. 1, FIG. 1 is a schematic diagram of one embodiment of a therapeutic breathing device 100 (and may also be referred to as breathing device 100, or simply device 100, or as a therapeutic breathing apparatus 100, breathing apparatus 100, or simply apparatus 100). At least in the illustrated embodiment, the breathing device 100 includes, among other components, a structure 102, a breathing port 104, a reservoir 106, a sensor 108, a set of indicators 110 (e.g., indicators 110A through 110n), and a power switch 112.

The structure 102 may include any suitable structure that is capable of enabling and/or allowing a user to perform the various embodiments of a therapeutic breathing method, as discussed elsewhere herein. The structure 102 may be formed of and/or include any suitable material(s) that is/are known or developed in the future capable of providing support for the various components and/or enabling/allowing the user to perform the various embodiments of a therapeutic breathing method, as discussed elsewhere herein.

Example suitable materials for the structure 102 can include, but are not limited to, a plastic, a polymer, a carbon fiber, a metal, and/or an alloy, etc., among other suitable materials that are possible, each of which is contemplated herein. In certain embodiments, the structure 102 includes and/or is formed of a plastic and/or plastic material, among other materials that are possible, each of which is contemplated herein.

As illustrated in FIG. 1, the structure 102 includes a top surface 102T. The structure 102 and/or the top surface 102T may include any suitable shape that is known or developed in the future. At least in the illustrated embodiment, the structure 102 and/or the top surface 102T includes a circular or generally circular shape defined by and/or bound by a perimeter 102PT.

The structure 102 and/or the top surface 102T may include any suitable dimensions that are known or developed in the future capable of providing support for the various components of the structure 102 and/or capable of enabling/allowing a user to perform the various embodiments of a therapeutic breathing method, as discussed elsewhere herein. At least in the illustrated embodiment, circular shape or substantially circular shape of the structure 102 and/or the top surface 102T includes a diameter D.

The diameter D of the structure 102 and/or the top surface 102T may include any suitable length that is known or developed in the future. In various embodiments, the diameter D includes a length in the range of about two (2) inches to about four (4) inches, among other diameter lengths that are smaller than 2 inches or larger than 4 inches that are possible, each of which is contemplated herein. In certain embodiments, the diameter D includes a length of about 2.75 inches, while in other embodiments, the diameter D includes a length of about 2.75 inches, among other diameter lengths that are smaller than about 2.75 inches or larger than about 3 inches that are possible, each of which is contemplated herein.

In various embodiments, the diameter D of the structure 102 and/or the top surface 102T includes a length that allows and/or enables the breathing device 100 to be a hand-held device. That is, the structure 102 and/or the top surface 102T includes a maximum length so that a user is able to use the breathing device 100 with a single hand.

In additional or alternative embodiments, the diameter D of the structure 102 and/or the top surface 102T includes a length that allows and/or enables the breathing device 100 to be concealable within the hand of a user. That is, the structure 102 and/or the top surface 102T includes a maximum length so that a user is able to conceal the breathing device 100 within a single hand (e.g., the breathing device 100 is not visible while the breathing device 100 is housed in the user's hand and the user's hand is closed around the breathing device 100).

In some embodiments, the top surface 102T is flat or substantially flat. In other embodiments, the top surface 102T can include a contoured, textured, curved, and/or non-flat surface, among other surfaces that are possible, each of which is contemplated herein.

A breathing port 104 may include any suitable shape and/or contour that is known or developed in the future that can enable and/or allow a user to breath air therethrough. In various embodiments, the breathing port 104 includes and/or defines an aperture that allows/enables a user to inhale and exhale air therethrough as portions of a therapeutic breathing method, as discussed elsewhere herein.

In certain embodiments, the breathing port 104 includes a mouthpiece 104M that is configured to allow/enable a user to place the user's lips and/or mouth thereon and breathe through the breathing port 104. In some embodiments, the shape of the mouthpiece resembles the shape of a human mouth.

A reservoir 106 may include any suitable structure that can house one or more adjuvant substances therein. In certain embodiments, the reservoir includes and/or defines a cavity in the top surface 102T of the structure 102, among other suitable structures that can house one or more adjuvants.

The reservoir 106 may include any suitable shape(s) that is/are known or developed in the future that can house one or more adjuvant substances therein. At least in the illustrated embodiment, the reservoir 106 includes a circular or substantially circular shape, among other shapes that are possible, each of which is contemplated herein. Further, the reservoir 106 may include any suitable dimension(s) (e.g., length, width, diameter, radius, depth, etc.) that is/are known or developed in the future that can allow/enable one or more adjuvant substances to be housed in the reservoir 106.

In various embodiments, the reservoir 106 includes a lid 106L configured to cover the cavity of the reservoir 106. At least in the illustrated embodiment, the lid 106L includes a set of one or more apertures or holes, which can include any suitable quantity of apertures/holes that can allow/enable the adjuvant to escape/diffuse/emit from the reservoir 106 so that the user can ingest the adjuvant while performing a therapeutic breathing method.

In some embodiments, the one or more adjuvant substances include a liquid and/or semi-liquid. In additional or alternative embodiments, the one or more adjuvants include a solid and/or semi-solid.

An adjuvant can include any substance that can enhance the effectiveness of a therapeutic breathing method. An adjuvant, in various embodiments, can include a fragrance-emitting substance and/or a scent-emitting substance, which can include any suitable fragrance-emitting substance and/or a scent-emitting substance or type of fragrance-emitting substance and/or a scent-emitting substance that is known or developed in the future capable of emitting a fragrance and/or scent that can enhance the effectiveness of a therapeutic breathing method when inhaled/consumed in conjunction with the therapeutic breathing method.

In certain embodiments, the adjuvant includes one or more essential oils, which can include any suitable essential oil(s) that is/are known or developed in the future capable of enhance the effectiveness of a therapeutic breathing method when inhaled/consumed in conjunction with the therapeutic breathing method. Example, essential oils can include, but are not limited to, lemon oil, peppermint oil, lavender oil, tee tree oil, eucalyptus oil, clove oil, chamomile oil, orange oil, spearmint oil, rosemary oil, grapefruit oil, oregano oil, ginger oil, lemongrass oil, spruce oil, pine oil, sandalwood oil, cedarwood oil, marjoram oil, clary sage oil, valerian oil, patchouli oil, vetiver oil, ylang ylang oil, frankincense oil, myrrh oil, neroli oil, and bergamot oil, among other essential oils that are possible, each of which is contemplated herein.

A sensor 108 may include any suitable device, apparatus, and/or components that can sense the presence of a user. In certain embodiments, the sensor 108 is configured to detect that a user is within a predetermined distance of the sensor 108. For example, the sensor 108 can be configured to detect when a user is sufficiently proximate to the sensor 108 such that the user's lips/mouth is/are likely engaged with the breathing port 104 and/or mouthpiece 106M.

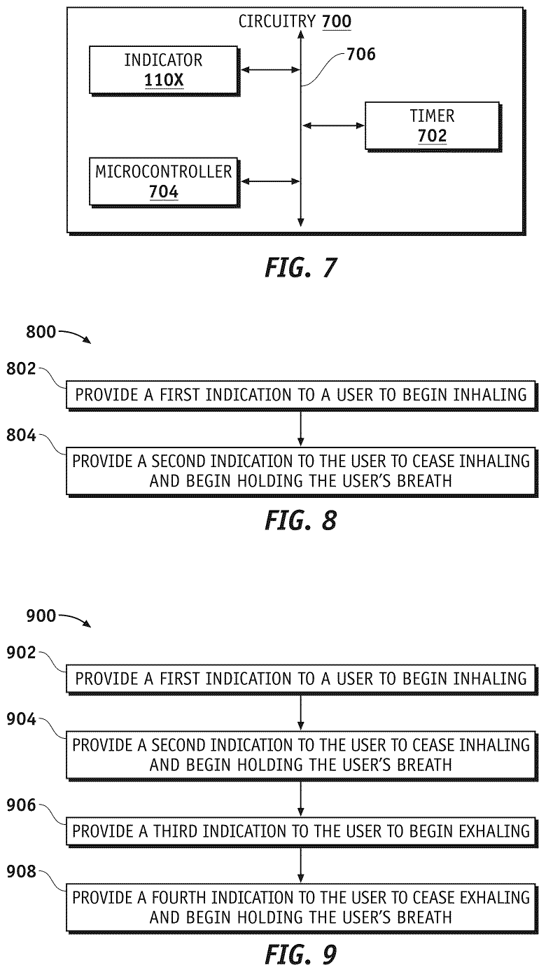

In various embodiments, the sensor 108 includes a timer 702 (see, FIG. 7) and is configured to initiate a therapeutic breathing method in response to detecting the presence of a user, as discussed elsewhere herein. In additional or alternative embodiments, the sensor 108 is in communication with the timer 702 and is configured to initiate a therapeutic breathing method in response to detecting the presence of a user, as discussed elsewhere herein.

A set of indicators 110 may include any suitable quantity of indicators 110 that is known or developed in the future that can assist and/or facilitate a user performing a therapeutic breathing method. At least in the illustrated embodiment, the set of indicators 110 includes five (5) indicators 110 (e.g., indicators 110A through 110n).

In various embodiments, a breathing pattern in a therapeutic breathing method can correspond to the quantity of indicators 110 included in the breathing device 100. For example, the quantity of indicators 110 can assist and/or facilitate the user performing a particular breathing pattern set forth in the therapeutic breathing method, as discussed elsewhere herein.

An indicator 110 may include any suitable cue(s) and/or type of cue(s) that is/are capable of assisting and/or facilitating the user performing a particular breathing pattern set forth in a therapeutic breathing method. Example cues and/or types of cues can include, but are not limited to, a visual cue, a tactile cue, and an auditory cue, among other cues and/or types of cues that are possible, each of which is contemplated herein.

A visual cue can include any suitable cue(s) and/or type of cue(s) that is/are known or developed in the future that a user can see/visualize. In some embodiments, a visual cue includes a visual cue generated by a light. Example visual cues and/or types of visual cues generated by a light can include, but are not limited to, one or more colors of light, one or more intensities of light (e.g., high intensity (bright/full), medium intensity (common/office), low intensity (dull/twilight), OFF (dark/night), etc.), one or more lengths of time a light is illuminated, and/or one or more patterns of light (e.g., solid, flashing, blinking, sequential, increasing/decreasing intensity, random, etc.), etc., among other visual cues and/or types of visual cues that can be generated by a light that are possible, each of which is contemplated herein.

A visual cue may be generated/displayed by an indicator 110, as discussed else wherein. In certain embodiments, the visual indicator 110 can be located on the top surface 102T, as illustrated in FIG. 1.

A tactile cue can include any suitable cue(s) and/or type of cue(s) that is/are known or developed in the future that a user can feel/sense by touch. In some embodiments, a tactile cue includes a cue generated by a vibration generating device/mechanism/component. Example tactile cues and/or types of tactile cues can include, but are not limited to, one or more vibrations (shakes), one or more intensities of vibration, one or more lengths of time a vibration occurs, and/or one or more patterns of vibration, etc., among other tactile cues and/or types of tactile cues that are possible, each of which is contemplated herein.

A tactile cue may be generated by an indicator 110, as discussed else wherein. In certain embodiments, the tactile indicator 110can be located on the top surface 102T as illustrated in FIG. 1 and/or internal to the structure 102, as discussed elsewhere herein.

An auditory cue can include any suitable cue(s) and/or type of cue(s) that is/are known or developed in the future that a user can hear and/or is audible. In some embodiments, an auditory cue includes a visual cue generated by a speaker. Example auditory cues and/or types of auditory cues can include, but are not limited to, one or more sounds, one or more tones, one or more sound intensities, one or more volumes, one or more pitches, one or more frequencies, one or more lengths of time a sound is audible, and/or one or more patterns of sounds, etc., among other auditory cues and/or types of auditory cues that are possible, each of which is contemplated herein.

An auditory cue may be generated by an indicator 110, as discussed else wherein. In certain embodiments, the auditory indicator 110 can be located on the top surface 102T, as illustrated in FIG. 1.

In the illustrated embodiment, the indicator 110A can include and/or provide one or more status indications of a therapeutic breathing method. Example status indications can include, but are not limited to, a ready/start indication, an in-progress indication, and/or a completed/finished indication, etc., among other status indications that are possible, each of which is contemplated herein.

The indicator 110A may include any one or more of the indicators 110 and/or one or more of the types of an indicator 110 discussed elsewhere herein. In certain embodiments, the indicator 110A can include a light that, when illuminated, visually provides a set of one or more status indications to a user.

In a non-limiting example, the indicator 110A can provide a first color (e.g., green) to indicate that the breathing device 100 is ready to begin a therapeutic breathing method, provide a second color (e.g., blue) to indicate that the therapeutic breathing method is in progress, and then provide a third color (e.g., red) or shut OFF to indicate that the therapeutic breathing method has ended. In another non-limiting example, the indicator 110A can provide a first color intensity to indicate that the breathing device 100 is ready to begin a therapeutic breathing method, provide a second color intensity to indicate that the therapeutic breathing method is in progress, and then provide a third color intensity or shut OFF to indicate that the therapeutic breathing method has ended. In still another non-limiting example, the indicator 110A can provide a first visual pattern (e.g., slowly blinking/flashing) to indicate that the breathing device 100 is ready to begin a therapeutic breathing method, provide a second visual pattern (e.g., rapidly blinking/flashing) to indicate that the therapeutic breathing method is in progress, and then provide a third visual pattern (e.g., solid color) or shut OFF to indicate that the therapeutic breathing method has ended.

While the above embodiments of the indicator 110A provide specific examples of the types and operations of an indicator 110A, the various embodiments of the indicator 110A are not limited to these examples. That is, the various embodiments of the indicator 110A can include different visual cues, a different quantity of visual cues, different status indications, and/or a different quantity of status indications, etc., as discussed herein, each of which is contemplated herein. Further, the various embodiments of the indicator 110A can include different types of cues (e.g., tactile cue, auditory cue, etc.), a different quantity of cues, different status indications for the cue(s), and/or a different quantity of status indications for the cue(s), etc., as discussed herein, each of which is contemplated herein. Moreover, the various embodiments of the indicator 110A can include two or more different cues (e.g., a visual cue and tactile cue, a visual cue and auditory cue, a tactile cue and auditory cue, or a visual cue, tactile cue, and auditory cue, etc.), different quantities of cues, different status indications for the cue(s), and/or a different quantity of status indications for the cue(s), etc., as discussed herein, each of which is contemplated herein.

The indicators 110B through 110n may each include any one or more of the indicators 110 and/or one or more of the types of an indicator 110 discussed elsewhere herein. In certain embodiments, the indicators 110B through 110n are each the same type of indicator 110 (e.g., a visual indicator, a tactile indicator, or an auditory indicator, etc.).

In various embodiments, the indicators 110B through 110n each include a light. When illuminated, each of indicators 110B through 110n can provide a visual cue to a user. The visual cues can be used to facilitate/assist the user in performing a therapeutic breathing method.

In certain embodiments, the indicators 110B through 110n are lights that can sequentially illuminate or turn OFF/extinguish. The indicators 110B through 110n can sequentially illuminate or turn OFF/extinguish in any suitable order that is known or developed in the future. For example, the indicators 110B through 110n can sequentially illuminate or turn OFF/extinguish in order (e.g., the indicator 110B can illuminate/extinguish first, followed by the indicator 110C, then the indicator 110D, and finally the indicator 110n), in reverse order (e.g., the indicator 110n can illuminate/extinguish first, followed by the indicator 110D, then the indicator 110C, and finally the indicator 110B), or in a random order (e.g., the indicator 110D can illuminate/extinguish first, followed by the indicator 110n, then the indicator 110C, and finally the indicator 110B).

The indicators 110B through 110n, in some embodiments, can sequentially illuminate (or turn OFF/extinguish) to guide a user through a first part/portion of one embodiment a therapeutic breathing method (e.g., an inhalation portion of the therapeutic breathing method, as discussed elsewhere herein), then sequentially illuminate (or turn OFF/extinguish) to guide a user through a second part/portion of the therapeutic breathing method (e.g., a breath holding portion of the therapeutic breathing method, as discussed elsewhere herein), and then further sequentially illuminate (or turn OFF/extinguish) to guide a user through a third part/portion of the therapeutic breathing method (e.g., an exhalation portion of the therapeutic breathing method, as discussed elsewhere herein).

In other embodiments, the indicators 110B through 110n can sequentially change colors to guide a user through a therapeutic breathing method. For example, the indicators 110B through 110n can be initially illuminated in a first color (e.g., blue) and then sequentially change from the first color to a second color (e.g., green) to guide a user through a first part/portion of one embodiment a therapeutic breathing method (e.g., an inhalation portion of the therapeutic breathing method, as discussed elsewhere herein), then sequentially change colors from the first color to the second color to guide a user through a second part/portion of the therapeutic breathing method (e.g., a breath holding portion of the therapeutic breathing method, as discussed elsewhere herein), and then further sequentially change colors from the first color to the second color to guide a user through a third part/portion of the therapeutic breathing method (e.g., an exhalation portion of the therapeutic breathing method, as discussed elsewhere herein).

In some embodiments, the indicators 110B through 110n each include the same color. In other embodiments, at least two of the indicators 110B through 110n include different colors. In still other embodiments, each of the indicators 110B through 110n includes a different color.

In certain embodiments, the indicators 110B through 110n each illuminate the same color (e.g., blue, green, yellow, red, purple, orange, white, gray, etc.) during all portions of the therapeutic breathing method. In other embodiments, the indicators 110B through 110n each illuminate the same first color (e.g., green, etc.) during the first portion of the therapeutic breathing method, illuminate the same second color (e.g., yellow, etc.) that is different than the first and third colors during the second portion of the therapeutic breathing method, and illuminate the same third color (e.g., blue, etc.) that is different than the first and second colors during the third portion of the therapeutic breathing method.

In other embodiments, the indicators 110B through 110n are lights that can sequentially increase in illuminated intensity. The indicators 110B through 110n can sequentially increase in intensity in any suitable order (e.g., chronological order, reverse order, random order, etc.) that is known or developed in the future, similar to the embodiments discussed above.

For example, the indicators 110B through 110n can be initially illuminated and then have their intensities sequentially increased to guide a user through a first part/portion of one embodiment a therapeutic breathing method (e.g., an inhalation portion of the therapeutic breathing method, as discussed elsewhere herein), then sequentially increase their respective intensities to guide a user through a second part/portion of the therapeutic breathing method (e.g., a breath holding portion of the therapeutic breathing method, as discussed elsewhere herein), and then further sequentially increase their respective intensities to guide a user through a third part/portion of the therapeutic breathing method (e.g., an exhalation portion of the therapeutic breathing method, as discussed elsewhere herein).

In still other embodiments, the indicators 110B through 110n are lights that can sequentially illuminate a pattern of light. The indicators 110B through 110n can sequentially illuminate a pattern of light in any suitable order (e.g., chronological order, reverse order, random order, etc.) that is known or developed in the future, similar to the embodiments discussed above.

For example, the indicators 110B through 110n can be initially illuminated and then sequentially display a pattern (e.g., blinking, flashing, etc.) to guide a user through a first part/portion of one embodiment a therapeutic breathing method (e.g., an inhalation portion of the therapeutic breathing method, as discussed elsewhere herein), then sequentially display the pattern to guide a user through a second part/portion of the therapeutic breathing method (e.g., a breath holding portion of the therapeutic breathing method, as discussed elsewhere herein), and then further sequentially display a pattern to guide a user through a third part/portion of the therapeutic breathing method (e.g., an exhalation portion of the therapeutic breathing method, as discussed elsewhere herein).

In some embodiments, the displayed a pattern can be the same pattern in each portion of the therapeutic breathing method. In other embodiments, the different patterns are displayed in each portion of the therapeutic breathing method. For example, the pattern may include a first pattern (solidly lit) during the first portion, a second pattern (slowly flashing/blinking) during the second portion, and a third pattern (slowly flashing/blinking) during the third portion.

As discussed herein, the illumination (or turning OFF/extinguishing) of the indicators 110B through 110n individually during each portion of the therapeutic breathing method can define a visual pattern and/or a pattern of cues. Additionally, or alternatively, the illumination (or turning OFF/extinguishing) of the indicators 110B through 110n during the various portions of the therapeutic breathing method define a visual pattern and/or a pattern of cues.

In certain embodiments, the indicator 110A can include a vibrating mechanism that, when vibrating, visually provides a set of one or more status indications to a user. Here, the indicator 110A can be considered a tactile indicator.

In a non-limiting example, the indicator 110A can provide a first vibration to indicate that the breathing device 100 is ready to begin a therapeutic breathing method, provide a second vibration to indicate that the therapeutic breathing method is in progress, and then provide a third vibration to indicate that the therapeutic breathing method has ended. In another non-limiting example, the indicator 110A can provide a first vibration intensity to indicate that the breathing device 100 is ready to begin a therapeutic breathing method, provide a second vibration intensity to indicate that the therapeutic breathing method is in progress, and then provide a third vibration intensity to indicate that the therapeutic breathing method has ended. In still another non-limiting example, the indicator 110A can provide a first vibration pattern (e.g., constant vibration) to indicate that the breathing device 100 is ready to begin a therapeutic breathing method, provide a second vibration pattern (e.g., slow vibration) to indicate that the therapeutic breathing method is in progress, and then provide a third vibration pattern (e.g., fast vibration) or shut OFF to indicate that the therapeutic breathing method has ended.

While the above embodiments of the indicator 110A provide specific examples of the types and operations of an indicator 110A (e.g., a tactile indicator), the various embodiments of the indicator 110A are not limited to these examples. That is, the various embodiments of the indicator 110A can include different tactile cues, a different quantity of tactile cues, different status indications, and/or a different quantity of status indications, etc., as discussed herein, each of which is contemplated herein. Further, the various embodiments of the indicator 110A can include different types of cues (e.g., visual cue, auditory cue, etc.), a different quantity of cues, different status indications for the cue(s), and/or a different quantity of status indications for the cue(s), etc., as discussed herein, each of which is contemplated herein. Moreover, the various embodiments of the indicator 110A can include two or more different cues (e.g., a visual cue and tactile cue, a visual cue and auditory cue, a tactile cue and auditory cue, or a visual cue, tactile cue, and auditory cue, etc.), different quantities of cues, different status indications for the cue(s), and/or a different quantity of status indications for the cue(s), etc., as discussed herein, each of which is contemplated herein.

In various embodiments, the indicators 110B through 110n each include a vibration device/mechanism. When vibrating, each of indicators 110B through 110n can provide a tactile cue to a user. The tactile cues can be used to facilitate/assist the user in performing a therapeutic breathing method.

In certain embodiments, the indicators 110B through 110n are vibration devices that can sequentially vibrate. The indicators 110B through 110n can sequentially vibrate in any suitable order that is known or developed in the future. For example, the indicators 110B through 110n can sequentially vibrate in order (e.g., the indicator 110B can illuminate/extinguish first, followed by the indicator 110C, then the indicator 110D, and finally the indicator 110n), in reverse order (e.g., the indicator 110n can illuminate/extinguish first, followed by the indicator 110D, then the indicator 110C, and finally the indicator 110B), or in a random order (e.g., the indicator 110D can illuminate/extinguish first, followed by the indicator 110n, then the indicator 110C, and finally the indicator 110B).

The indicators 110B through 110n, in some embodiments, can sequentially vibrate to guide a user through a first part/portion of one embodiment a therapeutic breathing method (e.g., an inhalation portion of the therapeutic breathing method, as discussed elsewhere herein), then sequentially vibrate to guide a user through a second part/portion of the therapeutic breathing method (e.g., a breath holding portion of the therapeutic breathing method, as discussed elsewhere herein), and then further sequentially vibrate to guide a user through a third part/portion of the therapeutic breathing method (e.g., an exhalation portion of the therapeutic breathing method, as discussed elsewhere herein).

In some embodiments, the indicators 110B through 110n each include the same vibration. In other embodiments, at least two of the indicators 110B through 110n include different vibrations. In still other embodiments, each of the indicators 110B through 110n includes a different vibration.

In certain embodiments, the indicators 110B through 110n each include the same vibration during all portions of the therapeutic breathing method. In other embodiments, the indicators 110B through 110n each include the same first vibration during the first portion of the therapeutic breathing method, include the same second vibration that is different than the first and third vibrations during the second portion of the therapeutic breathing method, and include the same third vibration that is different than the first and second vibrations during the third portion of the therapeutic breathing method.

In other embodiments, the indicators 110B through 110n are vibration devices that can sequentially increase in vibration intensity. The indicators 110B through 110n can sequentially increase in intensity in any suitable order (e.g., chronological order, reverse order, random order, etc.) that is known or developed in the future, similar to the embodiments discussed above.

For example, the indicators 110B through 110n can be initially vibrating and then have their intensities sequentially increased to guide a user through a first part/portion of one embodiment a therapeutic breathing method (e.g., an inhalation portion of the therapeutic breathing method, as discussed elsewhere herein), then sequentially increase their respective intensities to guide a user through a second part/portion of the therapeutic breathing method (e.g., a breath holding portion of the therapeutic breathing method, as discussed elsewhere herein), and then further sequentially increase their respective intensities to guide a user through a third part/portion of the therapeutic breathing method (e.g., an exhalation portion of the therapeutic breathing method, as discussed elsewhere herein).

In still other embodiments, the indicators 110B through 110n are vibration devices that can sequentially provide a pattern of vibration. The indicators 110B through 110n can sequentially provide a pattern of vibration in any suitable order (e.g., chronological order, reverse order, random order, etc.) that is known or developed in the future, similar to the embodiments discussed above.

For example, the indicators 110B through 110n can be initially vibrating and then sequentially vibrate in a pattern (e.g., rapid vibration, slow vibration, long vibration, short vibration, etc.) to guide a user through a first part/portion of one embodiment a therapeutic breathing method (e.g., an inhalation portion of the therapeutic breathing method, as discussed elsewhere herein), then sequentially vibrate in the pattern to guide a user through a second part/portion of the therapeutic breathing method (e.g., a breath holding portion of the therapeutic breathing method, as discussed elsewhere herein), and then further sequentially vibrate in the pattern to guide a user through a third part/portion of the therapeutic breathing method (e.g., an exhalation portion of the therapeutic breathing method, as discussed elsewhere herein).

In some embodiments, the vibration pattern can be the same pattern in each portion of the therapeutic breathing method. In other embodiments, the different patterns are displayed in each portion of the therapeutic breathing method. For example, the pattern may include a first vibration pattern during the first portion, a second vibration pattern during the second portion, and a third vibration pattern during the third portion.

As discussed herein, the indicators 110B through 110n individually vibrating during each portion of the therapeutic breathing method can define a tactile pattern and/or a pattern of cues. Additionally, or alternatively, the indicators 110B through 110n vibrating during the various portions of the therapeutic breathing method define a tactile pattern and/or a pattern of cues.

In certain embodiments, the indicator 110A can include a speaker that, when emitting sound, audibly provides a set of one or more status indications to a user. Here, the indicator 110A can be considered an auditory indicator.

In various embodiments, the indicators 110B through 110n each include a speaker. When emitting sound/noise, each of indicators 110B through 110n can provide an auditory cue to a user. The auditory cues can be used to facilitate/assist the user in performing a therapeutic breathing method.

In certain embodiments, the indicators 110B through 110n are speakers that can sequentially emit sound or turn OFF/extinguish. The indicators 110B through 110n can sequentially emit sound or turn OFF/extinguish in any suitable order that is known or developed in the future. For example, the indicators 110B through 110n can sequentially emit sound or turn OFF/extinguish in order (e.g., the indicator 110B can emit sound/extinguish first, followed by the indicator 110C, then the indicator 110D, and finally the indicator 110n), in reverse order (e.g., the indicator 110n can emit sound/extinguish first, followed by the indicator 110D, then the indicator 110C, and finally the indicator 110B), or in a random order (e.g., the indicator 110D can emit sound/extinguish first, followed by the indicator 110n, then the indicator 110C, and finally the indicator 110B).

The indicators 110B through 110n, in some embodiments, can sequentially emit sound (or turn OFF/extinguish) to guide a user through a first part/portion of one embodiment a therapeutic breathing method (e.g., an inhalation portion of the therapeutic breathing method, as discussed elsewhere herein), then sequentially emit sound (or turn OFF/extinguish) to guide a user through a second part/portion of the therapeutic breathing method (e.g., a breath holding portion of the therapeutic breathing method, as discussed elsewhere herein), then further sequentially emit sound (or turn OFF/extinguish) to guide a user through a third part/portion of the therapeutic breathing method (e.g., an exhalation portion of the therapeutic breathing method, as discussed elsewhere herein), and then sequentially emit sound (or turn OFF/extinguish) to guide a user through a fourth part/portion of the therapeutic breathing method (e.g., a breath holding portion of the therapeutic breathing method, as discussed elsewhere herein).

In other embodiments, the indicators 110B through 110n can sequentially change sounds to guide a user through a therapeutic breathing method. For example, the indicators 110B through 110n can be initially emit in a first sound and then sequentially change from the first sound to a second sound (e.g., green) to guide a user through a first part/portion of one embodiment a therapeutic breathing method (e.g., an inhalation portion of the therapeutic breathing method, as discussed elsewhere herein), then sequentially change sounds from the first sound to the second sound to guide a user through a second part/portion of the therapeutic breathing method (e.g., a breath holding portion of the therapeutic breathing method, as discussed elsewhere herein), then further sequentially change sounds from the first sound to the second sound to guide a user through a third part/portion of the therapeutic breathing method (e.g., an exhalation portion of the therapeutic breathing method, as discussed elsewhere herein), and then sequentially change sounds from the first sound to the second sound to guide a user through a fourth part/portion of the therapeutic breathing method (e.g., a breath holding portion of the therapeutic breathing method, as discussed elsewhere herein).

In some embodiments, the indicators 110B through 110n each include the same sound. In other embodiments, at least two of the indicators 110B through 110n include different sounds. In still other embodiments, each of the indicators 110B through 110n includes a different sound.

In certain embodiments, the indicators 110B through 110n each emit the same sound during all portions of the therapeutic breathing method. In other embodiments, the indicators 110B through 110n each emit the same first sound during the first portion of the therapeutic breathing method, emit the same second sound that is different than the first and third sounds during the second portion of the therapeutic breathing method, and emit the same third sound that is different than the first and second sounds during the third portion of the therapeutic breathing method.

In other embodiments, the indicators 110B through 110n are speakers that can sequentially increase in sound intensity. The indicators 110B through 110n can sequentially increase in intensity in any suitable order (e.g., chronological order, reverse order, random order, etc.) that is known or developed in the future, similar to the embodiments discussed above.

For example, the indicators 110B through 110n can initially emit a sound and then have their volumes/tones/pitches/frequencies sequentially increased to guide a user through a first part/portion of one embodiment a therapeutic breathing method (e.g., an inhalation portion of the therapeutic breathing method, as discussed elsewhere herein), then sequentially increase their respective volumes/tones/pitches/frequencies to guide a user through a second part/portion of the therapeutic breathing method (e.g., a breath holding portion of the therapeutic breathing method, as discussed elsewhere herein), then further sequentially increase their respective volumes/tones/pitches/frequencies to guide a user through a third part/portion of the therapeutic breathing method (e.g., an exhalation portion of the therapeutic breathing method, as discussed elsewhere herein), and then sequentially increase their respective volumes/tones/pitches/frequencies to guide a user through a fourth part/portion of the therapeutic breathing method (e.g., a breath holding portion of the therapeutic breathing method, as discussed elsewhere herein).

In still other embodiments, the indicators 110B through 110n are speakers that can sequentially emit a pattern of sound. The indicators 110B through 110n can sequentially emit a pattern of sound in any suitable order (e.g., chronological order, reverse order, random order, etc.) that is known or developed in the future, similar to the embodiments discussed above.

For example, the indicators 110B through 110n can emit an initial sound and then sequentially display a pattern of sound to guide a user through a first part/portion of one embodiment a therapeutic breathing method (e.g., an inhalation portion of the therapeutic breathing method, as discussed elsewhere herein), then sequentially emit the pattern of sound to guide a user through a second part/portion of the therapeutic breathing method (e.g., a breath holding portion of the therapeutic breathing method, as discussed elsewhere herein), then further sequentially emit a pattern of sound to guide a user through a third part/portion of the therapeutic breathing method (e.g., an exhalation portion of the therapeutic breathing method, as discussed elsewhere herein), and then sequentially emit the pattern of sound to guide a user through a fourth part/portion of the therapeutic breathing method (e.g., a breath holding portion of the therapeutic breathing method, as discussed elsewhere herein).

In some embodiments, the emitted pattern can be the same pattern in each portion of the therapeutic breathing method. In other embodiments, the different patterns of sound are emitted in each portion of the therapeutic breathing method. For example, the pattern may include a first sound pattern during the first portion, a second pattern during the second portion, and a third pattern during the third portion.

As discussed herein, the emission of sound (or turning OFF/extinguishing) of the indicators 110B through 110n individually during each portion of the therapeutic breathing method can define an auditory pattern and/or a pattern of cues. Additionally, or alternatively, the emission of sound (or turning OFF/extinguishing) of the indicators 110B through 110n during the various portions of the therapeutic breathing method define an auditory pattern and/or a pattern of cues.

While the above embodiments of the indicators 110B, 110C, 110D, and/or 110n provide specific examples of the type and operations of the indicators 110B, 110C, 110D, and/or 110n, the various embodiments of the indicators 110B, 110C, 110D, and/or 110n are not limited to these examples. That is, the various embodiments of the indicators 110B, 110C, 110D, and/or 110n can include different cues, different types of cues, a different quantity of cues, etc., as discussed herein, each of which is contemplated herein.

Further, the various embodiments of the indicators 110B, 110C, 110D, and/or 110n can include different cues (e.g., tactile cue, auditory cue, etc.), a different quantity of cues, different status indications for the cue(s), and/or a different quantity of status indications for the cue(s), etc., as discussed herein, each of which is contemplated herein. Moreover, the various embodiments of the indicators 110B, 110C, 110D, and/or 110n can two or more different cues (e.g., a visual cue and tactile cue, a visual cue and auditory cue, a tactile cue and auditory cue, or a visual cue, tactile cue, and auditory cue, etc.), different quantities of cues, different status indications for the cue(s), and/or a different quantity of status indications for the cue(s), etc., as discussed herein, each of which is contemplated herein.

A power switch 112 may include any suitable device that is known or developed in the future that is capable of turning the breathing device 100 ON/OFF. That is, the power switch 112 may include any suitable circuitry that can power up and/or power down the breathing device 100.

FIG. 2 illustrates a bottom view of the breathing device 100. At least in the illustrated embodiment, the structure 102 includes a bottom surface 102B.

In some embodiments, the bottom surface 102B is flat or substantially flat. In other embodiments, the bottom surface 102B can include a contoured, textured, curved, and/or non-flat surface, among other surfaces that are possible, each of which is contemplated herein.

The bottom surface 102B may include any suitable shape that is known or developed in the future. At least in the illustrated embodiment, the bottom surface 102B includes a circular or generally circular shape defined by and/or bound by a perimeter 102PB.

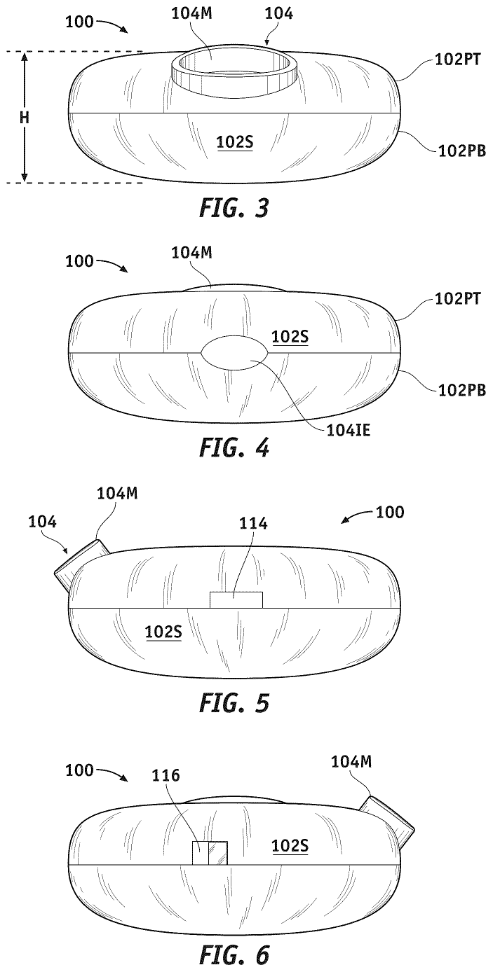

FIG. 3 is a front view of the breathing device 100. As illustrated in FIG. 3, the structure 102 includes a side surface 102s that extends between and around/along the perimeter 102 PT of the top surface 102T and the perimeter 102PB of the bottom surface 102B.

The side surface 102s includes a height H, which may include any suitable height that is known or developed in the future. In various embodiments, the height H of the side surface 102S includes a height H in the range of about one (1) inch to around three (3) includes, among other heights that are less than 1 inch and greater than 3 inches that are possible, each of which is contemplated herein.

FIG. 4 is a rear view of the breathing device 100. As illustrated, the breathing port 104 includes an intake/exhaust port 104IE that provides the user access to the air in the environment surrounding the breathing device 100 as the user breathes through the breathing port 104 and/or intake/exhaust port 104IE. Here, the intake/exhaust port 104IE is in ventilation communication with the mouthpiece 104M such that the user is able to inhale/exhale air via the breathing port 104.

FIG. 5 is a side view of the breathing device 100. As illustrated, the breathing device 100 includes a charging port 114 that provides charge to a battery (see, FIG. 7) used to power the set of indicators 110. The charging port 114 may include any suitable charging port and/or type of charging port that is known or developed in the future. Example charging ports 114 can include, but are not limited to, UBS-A, USB-B, USB-C, Micro-USB, and Mini-USB, etc., among other charging ports and/or types of charging port that are possible, each of which is contemplated herein.

FIG. 6 is another side view of the breathing device 100 that is opposite the side illustrated in FIG. 5. As illustrated in FIG. 6, the breathing device 100 includes an indicator switch 114 that switches ON/OFF the set of indicators 110 and/or can switch between the type of the indicators 110. The indicator switch 114 may include any suitable switch and/or type of switch that is known or developed in the future.

In some embodiments, the indicator switch 114 can turn ON/OFF an indicator 110X that is internal and/or housed within the structure 102 (see, FIG. 7). In other embodiments, the indicator switch 114 can switch between using the set of indicators 110 on the top of the structure 102 and the indicator 110X that is internal and/or housed within the structure 102.

FIG. 7 is a schematic block diagram of one embodiment of circuitry 700 house in the structure 102 of the breathing device 100. At least in the illustrated embodiment, the circuitry 700 includes an indicator 110X, a timer 702, and a microcontroller 704 coupled to and/or in communication with one another via a bus 706 (e.g., a wired and/or wireless bus).

The indicator 110X may include any suitable indicator 110 that can provide a tactile cue as discussed elsewhere herein. That is, the indicator 110X can include any suitable device and/or mechanism that can vibrate similar to the various embodiments of a tactile indicator 110 for providing one or more tactile cues to the user.

A timer 702 may include any suitable device and/or circuitry capable of counting time, keeping time, and/or tracking time that is known or developed in the future. Here, the timer 702 is configured to count time to facilitate a user performing a therapeutic breathing method.

In various embodiments, the timer 702 is configured to count a predetermined quantity of time. The predetermined quantity of time may include any suitable block of time that is known or developed in the future. In some embodiments, the timer 702 is configured to count off seconds.

In some embodiments, the timer 702 is configured to count off a block of seconds. The block of seconds may include any suitable quantity of seconds that forms at least a portion of a therapeutic breathing method. In certain embodiments, the time is configured to count off a block of four (4) seconds, among other blocks of seconds that are greater than 4 seconds or less that 4 seconds that are possible, each of which is contemplated herein.

The timer 702 is configured to communicate the count to the microcontroller 704. That is, the timer is configured to send and receive signals with the microcontroller 704.

A microcontroller 704 may include any suitable hardware, software, and/or firmware that is known or developed in the future capable of performing the operations and/or functions discuss below. The microcontroller 704 is configured to communicate with the sensor 108, the set of indicators 110 (and/or the indicator 110X), and the timer 702.

The microcontroller 704 is configured to receive a signal from the sensor 108 indicating that the sensor 108 has detected the presence of a user. The microcontroller 704 is further configured to initiate a therapeutic breathing method in response to receiving the signal from the sensor 108.

In initiating the therapeutic breathing method, the microcontroller 704 is configured to illuminate the indicator 110A to notify the user that the therapeutic breathing method is about to begin. The microcontroller 704 is also configured to notify the timer 702 to begin counting and receive the count from the timer 702.

As the microcontroller 704 receives the count from the timer 702, the microcontroller 704 is configured to actuate the set of indicators 110 or cause the set of indicators 110 to be actuated so that the user is able to perform a therapeutic breathing method, as discussed elsewhere herein.

The following is a non-limiting example of one embodiment of the operations/functions of a microcontroller 704. The microcontroller 704 is configured to receive a signal from the sensor 108. In response to receiving the signal from the sensor 108, the microcontroller 704 readies the timer 702.

In response to the timer 702 being ready, the microcontroller 704 actuates the indicator 110A to notify the user that the therapeutic breathing method is ready to begin and then actuates the indicator 110A again to indicate that the therapeutic breathing method has begun. The microcontroller 704 then sequentially actuates the indicators 110B, 110C, 110D, and 110n in one second intervals. Here, the user is supposed to inhale for the duration of the four seconds. After the 4 seconds, the microcontroller 704 turns OFF the indicators 110B, 110C, 110D, and 110 n.

Next, the microcontroller 704 again sequentially actuates the indicators 110B, 110C, 110D, and 110n in one second intervals. Here, the user is supposed to hold the user's breath for the duration of the four seconds. After these 4 seconds, the microcontroller 704 again turns OFF the indicators 110B, 110C, 110D, and 110n.

Subsequently, the microcontroller 704 sequentially actuates the indicators 110B, 110C, 110D, and 110n in one second intervals. Here, the user is supposed to exhale for the duration of the four seconds.

Further, the microcontroller 704 again sequentially actuates the indicators 110B, 110C, 110D, and 110n in one second intervals. Here, the user is supposed to hold the user's breath for the duration of the four seconds. After these 4 seconds, the microcontroller 704 again turns OFF the indicators 110B, 110C, 110D, and 110n and actuates the indicator 110A to notify the user that the therapeutic breathing method has ended.

While a specific example of the operations of a microcontroller 704 have been presented above, this example is intended to assist in understanding the various operations/functions of the breathing device 100 and are not intended to limit the various embodiments in any way. That is, various other embodiments may actuate the set of indicators 110 in a different way, a different order, for different durations of time, and/or a different quantity of times consistent with the various embodiments discussed herein. That is, each possible combination of elements specifically disclosed herein and/or that may be inferred from the disclosure herein are considered be within the spirit and scope of the various embodiments of the breathing device 100.

FIG. 8 is a flow diagram of one embodiment of a therapeutic breathing method 800. At least in the illustrated embodiment, the method 800 can begin by the breathing device 100 (or microcontroller 704) providing a first indication to a user to begin inhaling (block 802).

The first indication may be provided by a set of one or more indicators 110 actuated by the breathing device 100 (or microcontroller 704). Further, the first indication may notify the user to inhale for a first predetermined amount of time as a first portion of a breathing pattern. In some embodiments, the first predetermined amount of time includes 4 seconds, among other amounts of time greater than or less than 4 seconds, as discussed elsewhere herein.

The method 800 further includes providing a second indication to the user to cease inhaling and begin holding the user's breath (block 804). The second indication may be provided by the indicator(s) 110. Further, the second indication may notify the user to hold the user's breath for a second predetermined amount of time as a second portion of the breathing pattern. In some embodiments, the second predetermined amount of time includes 4 seconds, among other amounts of time greater than or less than 4 seconds, as discussed elsewhere herein.

The first and second indications may be any suitable set of one or more indications provided by indicator(s) 110A through 110n and/or 110X, as discussed elsewhere herein. That is, the first and second indications may include a visual indication, a tactile indication, and/or an auditory indication, as discussed elsewhere herein.

In some embodiments, the first and second indications are the same type of indication. In other embodiments, the first and second indications are different types of indications.

FIG. 9 is a flow diagram of another embodiment of a therapeutic breathing method 900. At least in the illustrated embodiment, the method 900 can begin by the breathing device 100 (or microcontroller 704) providing a first indication to a user to begin inhaling (block 902).

The first indication may be provided by a set of one or more indicators 110 actuated by the breathing device 100 (or microcontroller 704). Further, the first indication may notify the user to inhale for a first predetermined amount of time as a first portion of a breathing pattern. In some embodiments, the first predetermined amount of time includes 4 seconds, among other amounts of time greater than or less than 4 seconds, as discussed elsewhere herein.

The method 900 further includes providing a second indication to the user to cease inhaling and begin holding the user's breath (block 904). The second indication may be provided by the indicator(s) 110. Further, the second indication may notify the user to hold the user's breath for a second predetermined amount of time as a second portion of the breathing pattern. In some embodiments, the second predetermined amount of time includes 4 seconds, among other amounts of time greater than or less than 4 seconds, as discussed elsewhere herein.

In addition, the method 900 includes providing a third indication to the user to cease holding the user's breath and begin exhaling (block 906). The third indication may be provided by the indicator(s) 110. Further, the third indication may notify the user to exhale for a third predetermined amount of time as a third portion of the breathing pattern. In some embodiments, the second predetermined amount of time includes 4 seconds, among other amounts of time greater than or less than 4 seconds, as discussed elsewhere herein.

The method 900 further includes providing a fourth indication to the user to cease exhaling and begin holding the user's breath (block 908). The fourth indication may be provided by the indicator(s) 110. Further, the fourth indication may notify the user to hold the user's breath for a fourth predetermined amount of time as a fourth portion of the breathing pattern. In some embodiments, the fourth predetermined amount of time includes 4 seconds, among other amounts of time greater than or less than 4 seconds, as discussed elsewhere herein.

The first, second, third, and fourth indications may be any suitable set of one or more indications provided by indicator(s) 110A through 110n and/or 110X, as discussed elsewhere herein. That is, the first, second, and third indications may include a visual indication, a tactile indication, and/or an auditory indication, as discussed elsewhere herein.

In some embodiments, the first, second, third, and fourth indications are the same type of indication. In other embodiments, at least two of the first, second, third, and fourth indications are different types of indications. In still other embodiments, the first, second, third, and fourth indications are each different types of indications.

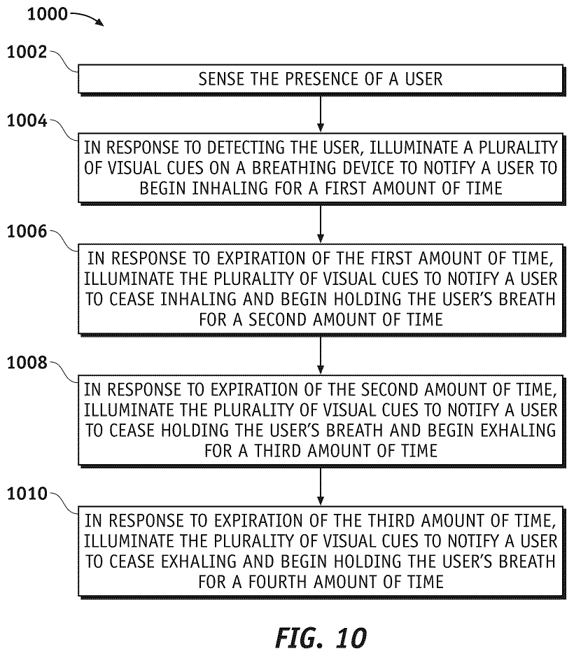

FIG. 10 is a flow diagram of a further embodiment of a therapeutic breathing method 1000. At least in the illustrated embodiment, the method 1000 can begin by a sensor 108 of a breathing device 100 sensing the presence of a user (block 1002).

The method 1000 further includes illuminating a plurality of visual cues on the breathing device 100 to notify the user to begin inhaling for a first predetermined amount of time as a first portion of a breathing pattern in response to the sensor 108 sensing the user (block 1004). The visual cues may include any embodiment of the visual cue(s) generated by a set of one or more indicators 110 discussed elsewhere herein. In some embodiments, the first predetermined amount of time includes 4 seconds, among other amounts of time greater than or less than 4 seconds, as discussed elsewhere herein.

In addition, the method 1000 includes illuminating the plurality of visual cues on the breathing apparatus to notify the user to cease inhaling and begin holding the user's breath for a second predetermined amount of time as a second portion of the breathing pattern in response to expiration of the first predetermined amount of time (block 1006). In some embodiments, the second predetermined amount of time includes 4 seconds, among other amounts of time greater than or less than 4 seconds, as discussed elsewhere herein.

Further, the method 1000 includes illuminating the plurality of visual cues on the breathing apparatus to notify the user to cease holding the user's breath and begin exhaling for a third predetermined amount of time as a third portion of the breathing pattern in response to expiration of the second predetermined amount of time (block 1008). In some embodiments, the third predetermined amount of time includes 4 seconds, among other amounts of time greater than or less than 4 seconds, as discussed elsewhere herein.

Further, the method 1000 includes illuminating the plurality of visual cues on the breathing apparatus to notify the user to cease exhaling and begin holding the user's breath for a fourth predetermined amount of time as a fourth portion of the breathing pattern in response to expiration of the third predetermined amount of time (block 1010). In some embodiments, the fourth predetermined amount of time includes 4 seconds, among other amounts of time greater than or less than 4 seconds, as discussed elsewhere herein.

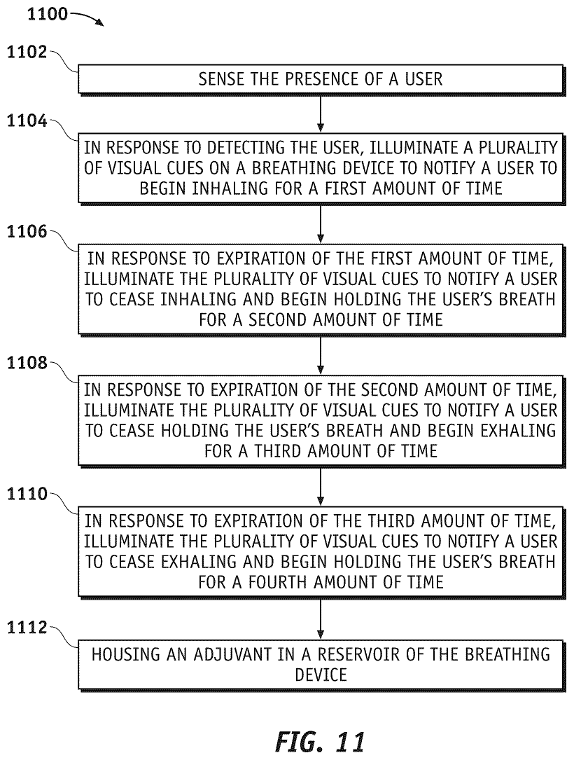

FIG. 11 is a flow diagram of another embodiment of a therapeutic breathing method 1100. At least in the illustrated embodiment, the method 1100 can begin by a sensor 108 of a breathing device 100 sensing the presence of a user (block 1102).

The method 1100 further includes illuminating a plurality of visual cues on the breathing device 100 to notify the user to begin inhaling for a first predetermined amount of time as a first portion of a breathing pattern in response to the sensor 108 sensing the user (block 1104). The visual cues may include any embodiment of the visual cue(s) generated by a set of one or more indicators 110 discussed elsewhere herein. In some embodiments, the first predetermined amount of time includes 4 seconds, among other amounts of time greater than or less than 4 seconds, as discussed elsewhere herein.

In addition, the method 1100 includes illuminating the plurality of visual cues on the breathing apparatus to notify the user to cease inhaling and begin holding the user's breath for a second predetermined amount of time as a second portion of the breathing pattern in response to expiration of the first predetermined amount of time (block 1106). In some embodiments, the second predetermined amount of time includes 4 seconds, among other amounts of time greater than or less than 4 seconds, as discussed elsewhere herein.

Further, the method 1100 includes illuminating the plurality of visual cues on the breathing apparatus to notify the user to cease holding the user's breath and begin exhaling for a third predetermined amount of time as a third portion of the breathing pattern in response to expiration of the second predetermined amount of time (block 1108). In some embodiments, the third predetermined amount of time includes 4 seconds, among other amounts of time greater than or less than 4 seconds, as discussed elsewhere herein.

Further, the method 1000 includes illuminating the plurality of visual cues on the breathing apparatus to notify the user to cease exhaling and begin holding the user's breath for a fourth predetermined amount of time as a fourth portion of the breathing pattern in response to expiration of the third predetermined amount of time (block 1110). In some embodiments, the fourth predetermined amount of time includes 4 seconds, among other amounts of time greater than or less than 4 seconds, as discussed elsewhere herein.

The method 1100 further includes housing an adjuvant in a reservoir 106 of the breathing device 100 (block 1112). The adjuvant may include any embodiment of one or more of the adjuvants discussed elsewhere herein.

In certain embodiments, the adjuvant includes an essential oil, as discussed elsewhere herein. In additional or alternative embodiments, the adjuvant includes a scent-emitting and/or a fragrance-emitting material and/or substance, as discussed elsewhere herein.

Embodiments may be practiced in other specific forms. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An apparatus, comprising:
a circular structure comprising:
a top surface including a top perimeter and a diameter;
a bottom surface including a bottom perimeter and the diameter, and
a curved surface including a height extending between the top perimeter and the bottom perimeter, wherein the height of the curved surface is less than the diameter; and
a breathing port formed in the circular structure for performing inhalations and exhalations as portions of a breathing pattern.

2. The apparatus of claim 1, further comprising a sensor configured to detect a presence of a user.

3. The apparatus of claim 2, further comprising:
a timer configured to facilitate the user following the breathing pattern; and
an indicator configured to notify the user of the breathing pattern,
wherein the indicator includes:
a first indication to notify the user when to begin inhaling in the breathing pattern,
a second indication to notify the user when to begin holding the user's breath,
a third indication to notify the user when to begin exhaling in the breathing pattern, and
a fourth indication to notify the user when to begin holding the user's breath.

4. The apparatus of claim 3, wherein:
the first indication, the second indication, the third indication, and the fourth indication include visual indications; and
at least two of the first indication, the second indication, the third indication, and the fourth indication include different visual indications.

5. The apparatus of claim 3, wherein:
the first indication, the second indication, the third indication, and the fourth indication include tactile indications; and
at least two of the first indication, the second indication, the third indication, and the fourth indication include different tactile indications.

6. The apparatus of claim 3, wherein:
the first indication, the second indication, the third indication, and the fourth indication include auditory indications; and
at least two of the first indication, the second indication, the third indication, and the fourth indication include different auditory indications.

7. The apparatus of claim 1, further comprising a reservoir configured to house an adjuvant.

8. The apparatus of claim 7, wherein the adjuvant comprises one of an essential oil, a fragrance-emitting substance, or a scent-emitting substance.

9. The apparatus of claim 8, wherein:
the adjuvant comprises the essential oil; and
the essential oil comprises at least one of lemon oil, peppermint oil, lavender oil, tee tree oil, eucalyptus oil, clove oil, chamomile oil, orange oil, spearmint oil, rosemary oil, grapefruit oil, oregano oil, ginger oil, lemongrass oil, spruce oil, pine oil, sandalwood oil, cedarwood oil, marjoram oil, clary sage oil, valerian oil, patchouli oil, vetiver oil, ylang ylang oil, frankincense oil, myrrh oil, neroli oil, or bergamot oil.

10. The apparatus of claim 1, wherein the circular structure comprises a size that is concealable within a hand of a user.

11. A method, comprising:
providing a first indication, by a breathing apparatus, to a user to begin inhaling for a first predetermined amount of time as a first portion of a breathing pattern; and
providing a second indication, by the breathing apparatus, to the user to cease inhaling and begin holding the user's breath for a second predetermined amount of time as a second portion of the breathing pattern, wherein:
the breathing apparatus comprises:
a circular structure comprising:
a top surface including a top perimeter and a diameter,
a bottom surface including a bottom perimeter and the diameter, and
a curved surface including a height extending between the top perimeter and the bottom perimeter, wherein the height of the curved surface is less than the diameter; and
a breathing port formed in the circular structure for performing inhalations and exhalations as portions of a breathing pattern, and
the user inhales via the breathing port.

12. The method of claim 11, further comprising:
providing a third indication, by the breathing apparatus, to the user to cease holding the user's breath and begin exhaling for a third predetermined amount of time as a third portion of the breathing pattern; and
providing a fourth indication, by the breathing apparatus, to the user to cease exhaling and begin holding the user's breath for a fourth predetermined amount of time as a fourth portion of the breathing pattern,
wherein the user exhales via the breathing port.

13. The method of claim 12, wherein the first predetermined amount of time, the second predetermined amount of time, the third predetermined amount of time, and the fourth predetermined amount of time include a same amount of time.

14. The method of claim 12, wherein at least two of the first predetermined amount of time, the second predetermined amount of time, the third predetermined amount of time, and the fourth predetermined amount of time include a different amount of time.

15. The method of claim 12, wherein:
providing the first indication comprises providing at least two of a visual indicator, a tactile indicator, and an auditory indicator;
providing the second indication comprises providing the at least two of the visual indicator, the tactile indicator, and the auditory indicator;
providing the third indication comprises providing the at least two of the visual indicator, the tactile indicator, and the auditory indicator; and
providing the fourth indication comprises providing the at least two of the visual indicator, the tactile indicator, and the auditory indicator.

16. The method of claim 12, wherein:
providing the first indication comprises providing one of a visual indicator, a tactile indicator, and an auditory indicator;
providing the second indication comprises providing the one of the visual indicator, the tactile indicator, and the auditory indicator;
providing the third indication comprises providing the one of the visual indicator, the tactile indicator, and the auditory indicator; and
providing the fourth indication comprises providing the one of the visual indicator, the tactile indicator, and the auditory indicator.

17. The method of claim 12, wherein:
providing the first indication comprises providing one of a visual indicator, a tactile indicator, and an auditory indicator;
providing the second indication comprises providing a first different one of the visual indicator, the tactile indicator, and the auditory indicator; and
providing one of the third indication or the fourth indication comprises providing a second different one of the visual indicator, the tactile indicator, and the auditory indicator.

18. A method, comprising:
sensing, by a sensor of a breathing apparatus, a presence of a user;
in response to sensing the user, illuminating a plurality of visual cues on the breathing apparatus to notify the user to begin inhaling for a first predetermined amount of time as a first portion of a breathing pattern;
in response to expiration of the first predetermined amount of time, illuminating the plurality of visual cues on the breathing apparatus to notify the user to cease inhaling and begin holding the user's breath for a second predetermined amount of time as a second portion of the breathing pattern; and
in response to expiration of the second predetermined amount of time, illuminating the plurality of visual cues on the breathing apparatus to notify the user to cease holding the user's breath and begin exhaling for a third predetermined amount of time as a third portion of the breathing pattern.

19. The method of claim 18, further comprising:
in response to expiration of the third predetermined amount of time, illuminating the plurality of visual cues on the breathing apparatus to notify the user to cease exhaling and begin holding the user's breath for a fourth predetermined amount of time as a fourth portion of the breathing pattern.

20. The method of claim 19, further comprising housing, in a reservoir of the breathing apparatus, a scent-emitting substance while the user is performing the breathing pattern.
